(19) United States
(12) Patent Application Publication
(10) Pub. No.: US 2026/0130606 A1
(43) Pub. Date: May 14, 2026

(54) APPARATUS AND A METHOD FOR PRODUCING INFORMATION INDICATIVE OF CARDIAC ABNORMALITY

(71) Applicant: Precordior Oy, Turku (FI)

(72) Inventors: Juuso BLOMSTER, Turku (FI); Mikko PÄNKÄÄLÄ, Turku (FI); Kamal DEHKORDI, Turku (FI); Tero HURNANEN, Turku (FI)

(21) Appl. No.: 19/429,954

(22) Filed: Dec. 22, 2025

Related U.S. Application Data
(63) Continuation of application No. PCT/EP2025/059398, filed on Apr. 7, 2025.

(30) Foreign Application Priority Data
Apr. 9, 2024 (FI) FI20247049
Apr. 9, 2024 (FI) FI20247050
Apr. 9, 2024 (FI) FI20247051

(51) Int. Cl. A61B 5/11; A61B 5/00

(52) U.S. Cl. CPC A61B 5/1126; A61B 5/6823; A61B 5/6898; A61B 5/7235; A61B 5/7275; A61B 2562/0219

(57) ABSTRACT

Described are computer-implemented methods for determining a cardiac abnormality of a subject. The computer-implemented methods may include obtaining a first signal indicative of cardiac acceleration. The computer-implemented methods may include obtaining a second signal indicative of cardiac rotation. The computer-implemented methods may include determining a first value relating to a property of the first signal and a second value relating to a property of the second signal. The computer-implemented methods may include generating an indication of cardiac abnormality of the subject based at least in part on a comparison between the first value and the second value. Described are systems configured to implement the computer-implemented methods.

### CROSS REFERENCE

This application is a continuation of International Patent Application No. PCT/EP2025/059398, filed Apr. 7, 2025, which claims the benefit of Finnish patent application No. FI20247049, filed Apr. 9, 2024, Finnish patent application No. FI20247050, filed Apr. 9, 2024, and Finnish patent application No. FI20247051, filed Apr. 9, 2024, each of which are incorporated by reference herein in their entirety.

### FIELD OF THE DISCLOSURE

The disclosure relates generally to producing information indicative of cardiac abnormality, such as aortic stenosis, heart valve disease, heart failure, atrial fibrillation, or other heart conditions. More particularly, the disclosure relates to an apparatus for producing information indicative of cardiac abnormality. Furthermore, the disclosure relates to methods, systems, and computer programs for producing information indicative of cardiac abnormality.

### BACKGROUND

Abnormalities that may occur in the cardiovascular system, if not diagnosed and appropriately treated and/or remedied, may progressively decrease the ability of the cardiovascular system to maintain a blood flow that meets the needs of a body of an individual especially when the individual encounters physical stress. For example, aortic stenosis occurs when the aortic valve narrows, and blood cannot flow normally. Aortic stenosis is typically caused by atherosclerosis, a calcium buildup on the aortic valve over time. These calcium deposits that often come with age make the valve tissue stiff, narrow, and unyielding. A condition of a patient may range from mild to severe. Over time, aortic valve stenosis causes the left ventricle of a heart to pump harder to push blood through the narrowed aortic valve. The extra effort may cause the left ventricle to thicken, enlarge, and weaken. If not diagnosed and appropriately treated and/or remedied, this form of heart valve disease may lead to a heart failure.

Heart failure occurs if the heart cannot pump or fill with blood adequately. Heart failure can be the result of stiffening, thickening, or thinning of the chambers of the heart, or from heart valve malfunctions. Heart failure can also occur from an infection or other disease that damages heart tissue.

Atrial fibrillation is an irregular, often rapid heart rate resulting from the atria of the heart beating out of sync with the ventricles. Atrial fibrillation can cause fatigue and can lead to blood clots, stroke, or even death, and can be lifelong.

Cardiovascular imaging techniques are typically used when abnormality of the aortic valve, such as aortic stenosis, is suspected. The cardiovascular imaging techniques include for example the following: transthoracic echocardiogram “TTE”, magnetic resonance imaging “MRI”, cardiac catheterization, transesophageal echocardiogram “TEE”, and computer tomography “CT” scanning. An inherent inconvenience related to imaging techniques of the kind mentioned above is that they typically require expensive equipment and specialized operating personnel. Therefore, there is a need for techniques for producing information indicative of cardiac abnormality, such as aortic stenosis, without a need for expensive equipment and specialized operating personnel.

### SUMMARY

The following presents a simplified summary to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a new apparatus for producing information indicative of cardiac abnormality, including but not limited to aortic stenosis, heart valve disease, heart failure, and atrial fibrillation. The apparatus according to the invention comprises:
a signal interface for receiving a first signal indicative of cardiac acceleration and measured with an accelerometer having a mechanical contact with a chest of an individual and for receiving a second signal indicative of cardiac rotation and measured with a gyroscope having a mechanical contact with the chest of the individual, and
a processing system coupled to the signal interface.

The processing system is configured to:
form a first descriptor value expressing a property of the first signal and a second descriptor value expressing the same property of the second signal, and
set an indicator signal outputted by the apparatus to express presence of cardiac abnormality, e.g. aortic stenosis, in response to a situation in which the first descriptor value and the second descriptor value with respect to each other are indicative of the cardiac abnormality.

The indicator signal can be transmitted to the individual and can be an instruction to seek treatment and/or advice from a healthcare provider. Alternatively or in combination, the indicator signal can be transmitted to a healthcare provider and can be an indication of the presence of a cardiac abnormality in the individual. The indicator signal can comprise cardiac measurement data transmitted to a healthcare provider.

In the light of empirical data, many cardiac abnormalities such as aortic stenosis affect differently the cardiac acceleration measured with an accelerometer than the cardiac rotation measured with a gyroscope. Thus, an indicator for cardiac abnormality can be formed by comparing a given property of the cardiac rotation to the same property of the cardiac acceleration. The properties of the cardiac rotation and the cardiac acceleration can be for example strengths, e.g. powers, energies, amplitudes, or the like of the cardiac rotation and the cardiac acceleration, or peak-to-average ratios of the cardiac rotation and the cardiac acceleration, or any other property which is affected differently by cardiac abnormalities in conjunction with the cardiac acceleration than in conjunction with the cardiac rotation.

It is to be noted that the situation in which the first and second descriptor values with respect to each other, i.e. when viewed in relation to each other, are indicative of the cardiac abnormality can be detected in many ways. For example, it is possible to check whether a ratio of the first and second descriptor values is outside a value-range corresponding to the healthy cases or whether the ratio is inside a value-range corresponding to cardiac abnormality cases, or to check whether an absolute value of a difference between the first and second descriptor values is above or below a limit normalized or scaled in accordance with the first and/or second descriptor values. It is also possible to deem the first and second descriptor values as coordinates of a geometric point in a two-dimensional geometric plane and to check whether this geometric point is outside a geometric area in the geometric plane corresponding to healthy cases or inside a geometric area corresponding to cardiac abnormality cases. Thus, the invention is not limited to any specific ways to detect the above-mentioned situation in which the first and second descriptor values with respect to each other, i.e. in relation to each other, are indicative of cardiac abnormality, e.g. aortic stenosis.

The apparatus may comprise a sensor system comprising an accelerometer and a gyroscope for measuring the above-mentioned signals indicative of the cardiac acceleration and the cardiac rotation. It is also possible that the signal interface is configured to receive the signals from an external device comprising an appropriate sensor system, i.e. it is emphasized that the apparatus does not necessarily comprise means for measuring the signals indicative of the cardiac acceleration and the cardiac rotation. The apparatus can be for example a smartphone or another hand-held device comprising a gyroscope and an accelerometer. The apparatus can be placed on an individual's chest to measure the above-mentioned signals caused by heartbeats. The apparatus can comprise, for example, a patch or wearable sensor able to contact the individual's chest when the individual is laying prone. The apparatus can be a smartphone, such as an Apple iPhone, an Android phone, a Google Pixel phone, Motorola phone, or another type of smartphone. The apparatus can comprise a medical provider device or other handheld medical device.

In this document, the term “gyroscope” covers sensors of various kinds for measuring angular rotations. A gyroscope can be for example a microelectromechanical system “MEMS” based on an effect of the Coriolis force acting on a back-and-forth turning object. In this document, the term “accelerometer” covers sensors of various kinds for measuring acceleration of a linear transverse motion. An accelerometer can be for example a microelectromechanical system “MEMS” based on the law of inertia. It is to be noted that the words “first” and “second” used in conjunction with the signals measured with an accelerometer and a gyroscope and also in conjunction with the descriptor values expressing a property of the accelerometer signal and the same property of the gyroscope signal are just labels and do not involve any indication concerning e.g. a measurement action or the like and that these labels can be interchanged or replaced with other labels e.g. “acc” and “gyro”, etc. without changing the subject matter.

The above-mentioned value-range corresponding to healthy cases can be determined based on empirical data gathered from a group of patients and healthy persons. The limit or limits of the value-range is/are not necessary constant or constants, but the limit or limits can be changing according to an individual under consideration, according to time, and/or according to some other factors. It is also possible to define many value-ranges each of which represents a specific probability of aortic stenosis or some other cardiac abnormality.

In accordance with the invention, there is also provided a new first computer-implemented system for detecting a cardiac abnormality or a risk of having a cardiac abnormality in an individual. The first computer-implemented system comprises:
a sensor apparatus for contacting a chest of the individual and configured to measure motion of the chest of the individual, and
a processing system configured to i) calculate one or more parameters relating to the cardiac abnormality from the measured motion of the chest of the individual and ii) detect a presence or a risk of having the cardiac abnormality based on the calculated one or more parameters, wherein the cardiac abnormality comprises aortic stenosis.

In the first computer-implemented system according to an exemplifying and non-limiting embodiment, the sensor apparatus further comprises an accelerometer, a gyroscope, or both.

In the first computer-implemented system according to an exemplifying and non-limiting embodiment, the sensor apparatus is provided in a smartphone.

In the first computer-implemented system according to an exemplifying and non-limiting embodiment, the measuring the motion of the chest of the individual comprises measuring an acceleration, a rotation, or both of a heart of the individual from the sensor apparatus placed on a chest of the individual.

In the first computer-implemented system according to an exemplifying and non-limiting embodiment, the one or more parameters describe cardiac acceleration, cardiac rotation, or both.

In the first computer-implemented system according to an exemplifying and non-limiting embodiment, the cardiac abnormality further comprises heart valve disease, heart failure, atrial fibrillation, or any combination thereof.

In accordance with the invention, there is also provided a new second computer-implemented system for detecting a cardiac abnormality or a risk of having a cardiac abnormality in an individual. The second computer-implemented system comprises:
a processing system configured to process accelerometer signal based on a measurement with an accelerometer and gyroscope signal based on a measurement with a gyroscope, wherein the processing comprises comparing the accelerometer signal and the gyroscope signal to one or more adjustable predetermined cardiac abnormality threshold data values, and
an output configured to make a signal available to a user, wherein the signal comprises an indication that the accelerometer signal and the gyroscope signal are above the one or more cardiac abnormality threshold data values.

The second computer-implemented system according to an exemplifying and non-limiting embodiment further comprises a sensor apparatus.

In the second computer-implemented system according to an exemplifying and non-limiting embodiment, the sensor apparatus further comprises an accelerometer, a gyroscope, or both.

In the second computer-implemented system according to an exemplifying and non-limiting embodiment, the accelerometer signal and the gyroscope signal are collected from the chest movement of the individual.

In the second computer-implemented system according to an exemplifying and non-limiting embodiment, the accelerometer signal further comprises one or more properties of a time-trend of acceleration measured from the individual.

In the second computer-implemented system according to an exemplifying and non-limiting embodiment, the gyroscope signal further comprises one or more properties of a time-trend of rotation measured from the individual.

In the second computer-implemented system according to an exemplifying and non-limiting embodiment, the processing system is further configured to compare the gyroscope signal to the accelerometer signal to produce a ratio output indicative of a ratio of the gyroscope signal to the accelerometer signal.

In the second computer-implemented system according to an exemplifying and non-limiting embodiment, the processing system is further configured to compare the one or more threshold values to the ratio output.

In the second computer-implemented system according to an exemplifying and non-limiting embodiment, the output further comprises an indication of a presence of cardiac abnormality, an indication of a risk of the cardiac abnormality, or an indication to contact a healthcare provider.

In the second computer-implemented system according to an exemplifying and non-limiting embodiment, the cardiac abnormality comprises aortic stenosis.

In accordance with the invention, there is also provided a new method for producing information indicative of cardiac abnormality, such as aortic stenosis. The method according to the invention comprises:
receiving a first signal indicative of cardiac acceleration and measured with an accelerometer having a mechanical contact with a chest of an individual,
receiving a second signal indicative of cardiac rotation and measured with a gyroscope having a mechanical contact with the chest of the individual,
forming a first descriptor value expressing a property of the first signal and a second descriptor value expressing the same property of the second signal, and
setting an indicator signal to express presence of cardiac abnormality, e.g. aortic stenosis, in response to a situation in which the first descriptor value and the second descriptor value with respect to each other are indicative of the cardiac abnormality.

In accordance with the invention, there is also provided a new first computer-implemented method for detecting a cardiac abnormality or a risk of having a cardiac abnormality in an individual. The first computer-implemented method comprises:
measuring, with a sensor apparatus placed on a chest of the individual, motion of a chest of the individual,
calculating one or more parameters related to the cardiac abnormality from the measured motion of the chest of the individual, and
detecting a presence or a risk of having the cardiac abnormality based on the calculated one or more parameters, wherein the cardiac abnormality comprises aortic stenosis.

In the first computer-implemented method according to an exemplifying and non-limiting embodiment, the sensor apparatus comprises an accelerometer, a gyroscope, or both.

In the first computer-implemented method according to an exemplifying and non-limiting embodiment, the sensor apparatus is provided in a smartphone.

In the first computer-implemented method according to an exemplifying and non-limiting embodiment, the measuring the motion of the chest of the individual comprises measuring an acceleration, a rotation, or both of a heart of the individual from the sensor apparatus placed on the chest.

In the first computer-implemented method according to an exemplifying and non-limiting embodiment, the one or more parameters describe cardiac acceleration, cardiac rotation, or both.

In the first computer-implemented method according to an exemplifying and non-limiting embodiment, the cardiac abnormality further comprises heart valve disease, heart failure, atrial fibrillation, or any combination thereof.

In accordance with the invention, there is also provided a new second computer-implemented method for detecting a cardiac abnormality or a risk of having a cardiac abnormality in an individual. The second computer-implemented method comprises:
processing accelerometer signal based on a measurement with an accelerometer and gyroscope signal based on a measurement with a gyroscope, wherein the processing comprises comparing the accelerometer signal and the gyroscope signal to one or more adjustable predetermined cardiac abnormality threshold data values, and
outputting a signal to the user, wherein the signal comprises an indication that the accelerometer signal and the gyroscope signal are above the one or more cardiac abnormality threshold data values.

The second computer-implemented method according to an exemplifying and non-limiting embodiment further comprises collecting the accelerometer signal and the gyroscope signal using a sensor apparatus.

In the second computer-implemented method according to an exemplifying and non-limiting embodiment, the accelerometer signal and the gyroscope signal are collected from the chest movement of the individual.

In the second computer-implemented method according to an exemplifying and non-limiting embodiment, the accelerometer signal further comprises one or more properties of a time-trend of acceleration measured from the individual.

In the second computer-implemented method according to an exemplifying and non-limiting embodiment, the gyroscope signal further comprises one or more properties of a time-trend of rotation measured from the individual.

In the second computer-implemented method according to an exemplifying and non-limiting embodiment, the processing further comprises comparing the gyroscope signal to the accelerometer signal to produce a ratio output indicative of a ratio of the gyroscope signal to the accelerometer signal.

The second computer-implemented method according to an exemplifying and non-limiting embodiment further comprises comparing the one or more threshold values to the ratio output.

The second computer-implemented method according to an exemplifying and non-limiting embodiment further comprises outputting an indication of a presence of cardiac abnormality, an indication of a risk of cardiac abnormality, or an indication to contact a healthcare provider.

In the second computer-implemented method according to an exemplifying and non-limiting embodiment, the cardiac abnormality comprises aortic stenosis.

In accordance with the invention, there is also provided a new computer program for producing information indicative of cardiac abnormality, such as aortic stenosis. The computer program comprises computer executable instructions for controlling a programmable processing system to:
receive a first signal indicative of cardiac acceleration and measured with an accelerometer having a mechanical contact with a chest of an individual,
receive a second signal indicative of cardiac rotation and measured with a gyroscope having a mechanical contact with the chest of the individual,
form a first descriptor value expressing a property of the first signal and a second descriptor value expressing the same property of the second signal, and
set an indicator signal to express presence of cardiac abnormality, e.g. aortic stenosis, in response to a situation in which the first descriptor value and the second descriptor value with respect to each other are indicative of the cardiac abnormality.

In accordance with the invention, there is provided also a new computer program product. The computer program product comprises a non-volatile computer readable medium, e.g. a compact disc “CD”, encoded with a computer program according to the invention.

A computer readable medium, such as computer-executable code, may take many forms, including but not limited to, a tangible storage medium, a carrier wave medium or physical transmission medium. Non-volatile storage media include, for example, optical or magnetic disks, such as any of the storage devices in any one or more computers or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency “RF” and infrared “IR” data communications. Common forms of computer-readable media therefore include for example: a floppy disk, a flexible disk, hard disk, magnetic tape, any other magnetic medium, a compact disc read only memory “CD-ROM”, digital video disc “DVD” or DVD-ROM, any other optical medium, punch cards paper tape, any other physical storage medium with patterns of holes, a random access memory “RAM”, a read only memory “ROM”, a programmable read only memory “PROM” and an erasable programmable read only memory “EPROM”, a flash-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Exemplifying and non-limiting embodiments are described in accompanied dependent claims.

Exemplifying and non-limiting embodiments both as to constructions and to methods of operation, together with additional objects and advantages thereof, will be best understood from the following description of specific exemplifying embodiments when read in conjunction with the accompanying drawings.

The verbs “to comprise” and “to include” are used in this document as open limitations that neither exclude nor require the existence of also un-recited features.

The features recited in the accompanied dependent claims are mutually freely combinable unless otherwise explicitly stated.

Furthermore, it is to be understood that the use of “a” or “an”, i.e. a singular form, throughout this document does not exclude a plurality.

The word “predetermined” means adjustably predetermined, advantageously able to be modified in real-time, and advantageously able to be adjusted by an individual whose cardiac condition is under consideration, a healthcare provider, and/or a third party.

### BRIEF DESCRIPTION OF FIGURES

Exemplifying and non-limiting embodiments and their advantages are explained in greater detail below with reference to the accompanying drawings, in which:

FIG. 1 shows a schematic illustration of an apparatus according to an exemplifying and non-limiting embodiment for producing information indicative of cardiac abnormality,

FIGS. 2a and 2b illustrate waveforms of exemplifying signals indicative of cardiac acceleration and cardiac rotation in a normal case when an individual under consideration is at rest,

FIGS. 3a and 3b illustrate waveforms of exemplifying signals indicative of cardiac acceleration and cardiac rotation in a case of aortic stenosis when an individual under consideration is at rest, and

FIG. 4 is a flow chart of a method according to an exemplifying and non-limiting embodiment for producing information indicative of cardiac abnormality.

### DESCRIPTION OF EXEMPLIFYING AND NON-LIMITING EMBODIMENTS

The specific examples provided in the description below should not be construed as limiting the scope and/or the applicability of the invention. Lists and groups of examples provided in the description are not exhaustive unless otherwise explicitly stated.

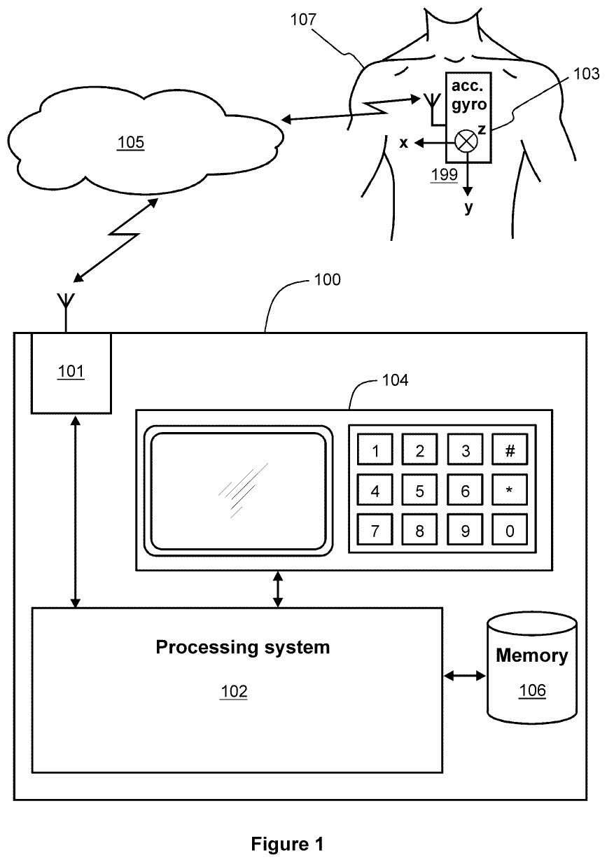

FIG. 1 shows a schematic illustration of an apparatus 100 according to an exemplifying and non-limiting embodiment for producing information indicative of cardiac abnormality such as aortic stenosis, heart valve disease, heart failure, and/or atrial fibrillation. The apparatus comprises a signal interface 101 for receiving a first signal indicative of cardiac acceleration and a second signal indicative of cardiac rotation. The apparatus 100 comprises a processing system 102 coupled to the signal interface 101. The processing system 102 is configured to:
form a first descriptor value expressing a property of the first signal and a second descriptor value expressing the same property of the second signal, and
set an indicator signal outputted by the apparatus 100 to express presence of cardiac abnormality in response to a situation in which the first descriptor value and the second descriptor value with respect to each other are indicative of the cardiac abnormality.

The above-mentioned first and second signals are produced with a sensor system 103 that comprises an accelerometer and a gyroscope. In the exemplifying situation shown in FIG. 1, the sensor system 103 is placed on the chest of an individual 107. The gyroscope and the accelerometer of the sensor system 103 can be separate devices, or the sensor system 103 may comprise for example an inertial measurement unit “IMU” comprising both an accelerometer and a gyroscope. The sensor system 103 can be for example a microelectromechanical system “MEMS”. The temporal duration of the first and second signals can be, for example but not necessarily, from tens of seconds to hours. The indicator signal outputted by the apparatus 100 can be for example a message shown on a display screen of a user-interface 104. It is also possible that the apparatus 100 contains the accelerometer and the gyroscope of the sensor system.

In the exemplifying case illustrated in FIG. 1, the sensor system 103 is connected to the signal interface 101 via one or more data transfer links each of which can be for example a radio link or a corded link. The data transfer from the sensor system 103 to the signal interface 101 may take place either directly or via a data transfer network 105 such as e.g. a telecommunication network. In the exemplifying case illustrated in FIG. 1, the sensor system 103 comprises a radio transmitter. It is also possible that the apparatus comprising the processing device 102 is integrated with the sensor system. In this exemplifying case, the signal interface is a simple wiring from the sensor system to the processing device. An apparatus comprising an integrated sensor system can be for example a smartphone or another hand-held device which can be placed on the chest of an individual during a measurement phase. The device can perform processing operations and contain the integrated sensor system. The integrated sensor system can comprise a gyroscope, an accelerometer, or both. In a case where the integrated sensor system does not comprise both the gyroscope and accelerometer, an external gyroscope or accelerometer can be used. The device can be for example a patch or wearable sensor able to contact the individual's chest when the individual is laying prone. The device can be a smartphone, such as an Apple iPhone, an Android phone, a Google Pixel phone, Motorola phone, or another type of smartphone. The device can be a medical provider device or other handheld medical device. The device can contain an integrated sensor system. It is also possible that the integrated sensor system comprises only an accelerometer or a gyroscope, and the device has a wireless or corded data interface for receiving a signal produced by an external gyroscope or accelerometer.

An apparatus according to an exemplifying and non-limiting embodiment of the invention is configured to record the first and second signals indicative of the cardiac acceleration and the cardiac rotation. The recorded signals can be measured within a time window having a fixed temporal start-point and a fixed temporal end-point, or within a sliding time window having a fixed temporal length and moving along with elapsing time. The apparatus may comprise an internal memory 106 for recording the signals and/or the apparatus may comprise a data port for connecting to an external memory. The apparatus may transmit and receive data wirelessly to and from an external memory.

There are numerous ways to define and form the first descriptor value expressing a property of the cardiac acceleration and the second descriptor value expressing the same property of the cardiac rotation. The first descriptor value may express for example the strength of the first signal, e.g. power, a root-mean-square “RMS” value, or the maximum amplitude of the first signal. Correspondingly, the second descriptor value can express for example the strength of the second signal, e.g. power, a root-mean-square “RMS” value, or the maximum amplitude of the second signal. Furthermore, the first descriptor value may express the peak-to-average ratio of the first signal and the second descriptor value may express the peak-to-average ratio of the second signal. Thus, the invention is not limited to any specific ways to define and form the first and second descriptor values.

In an apparatus according to an exemplifying and non-limiting embodiment, the processing system 102 is configured to form the first descriptor value Sacc to be proportional, e.g. directly proportional to energy of the first signal:

$${Σ}_{i = 1}^{N} ( {a}_{x i}^{2} + {a}_{y i}^{2} + {a}_{z i}^{2} ) , \qquad ( 1 )$$

and the second descriptor value Sᵣₒₜ to be proportional, e.g. directly proportional to energy of the second signal:

$${Σ}_{i = 1}^{N} ( {ω}_{x i}^{2} + {ω}_{y i}^{2} + {ω}_{z i}^{2} ) , \qquad ( 2 )$$

where i is an index increasing with time, N is the number of samples of the first and second signals on a time period under consideration, axi is an ith sample of an x-directional component of the cardiac acceleration in a cartesian coordinate system 199, ayi is an ith sample of a y-directional component of the cardiac acceleration in the cartesian coordinate system 199, azi is an ith sample of a z-directional component of the cardiac acceleration in the cartesian coordinate system 199, □xi is an ith sample of the cardiac rotation with respect to the x-direction of the cartesian coordinate system 199, □yi is an ith sample of the cardiac rotation with respect to the y-direction of the cartesian coordinate system 199, □zi is an ith sample of the cardiac rotation with respect to the z-direction of the cartesian coordinate system 199.

In an apparatus according to an exemplifying and non-limiting embodiment, the processing system 102 is configured to form the first descriptor value Sacc to be proportional, e.g. directly proportional to power of the first signal:

$${Σ}_{i = 1}^{N} ( {a}_{x i}^{2} + {a}_{y i}^{2} + {a}_{z i}^{2} ) / N , \qquad ( 3 )$$

and the second descriptor value Sᵣₒₜ to be proportional, e.g. directly proportional to power of the second signal:

$${Σ}_{i = 1}^{N} ( {ω}_{x i}^{2} + {ω}_{y i}^{2} + {ω}_{z i}^{2} ) / N . \qquad ( 4 )$$

In an apparatus according to an exemplifying and non-limiting embodiment, the processing system 102 is configured to form the first descriptor value S_{acc} to be proportional, e.g. directly proportional to:

$${Σ}_{i = 1}^{N} ( \vert {a}_{x i} \vert + \vert {a}_{y i} \vert + \vert {a}_{z i} \vert ) , \qquad ( 5 )$$

and the second descriptor value Sᵣₒₜ to be proportional, e.g. directly proportional to:

$${Σ}_{i = 1}^{N} ( \vert {ω}_{x i} \vert + \vert {ω}_{y i} \vert + \vert {ω}_{z i} \vert ) , \qquad ( 6 )$$

where |·| means the absolute value.

In an apparatus according to an exemplifying and non-limiting embodiment, the processing system 102 is configured to form the first descriptor value S_{acc} to be proportional, e.g. directly proportional to the maximum square amplitude of the first signal:

$$Max \{ ( {a}_{x i}^{2} + {a}_{y i}^{2} + {a}_{z i}^{2} \vert i = 1 , … , N \} , \qquad ( 7 )$$

and the second descriptor value Sᵣₒₜ to be proportional, e.g. directly proportional to the maximum square amplitude of the second signal:

$$Max \{ ( {ω}_{x i}^{2} + {ω}_{y i}^{2} + {ω}_{z i}^{2} \vert i = 1 , … , N \} . \qquad ( 8 )$$

In an apparatus according to an exemplifying and non-limiting embodiment, the processing system 102 is configured to form the first descriptor value S_{acc} to be proportional, e.g. directly proportional to the median value or the arithmetic average of peak values of the following signal X_{acc} on successive heart-beat periods, i.e. maximum values of X_{acc} during the heart-beat periods:

$${x}_{a c c} = \sqrt{( {a}_{x i}^{2}} + {a}_{yi}^{2} + {a}_{z i}^{2} ) , \qquad ( 9 )$$

and the second descriptor value Sᵣₒₜ to be proportional, e.g. directly proportional to the median value or the arithmetic average of peak values of the following signal Xᵣₒₜ on successive heart-beat periods, i.e. maximum values of Xᵣₒₜ during the heart-beat periods:

$${X}_{r o t} = \sqrt{( {ω}_{x i}^{2}} + {ω}_{y i}^{2} + {ω}_{z i}^{2} ) . \qquad ( 10 )$$

It is also possible that the above-mentioned X_{acc} is aₓᵢ²+a_{yi}²+a_{zi}² to avoid a need for computation of the square-root, and correspondingly Xᵣₒₜ is ωₓᵢ²+ω_{yj}²+ω_{zi}².

In an apparatus according to an exemplifying and non-limiting embodiment, the processing system 102 is configured to determine whether a ratio Sᵣₒₜ/S_{acc} of the first and second descriptor values is outside a predetermined value-range corresponding to healthy cases in order to determine whether the first and second descriptor values S_{acc} and S_{acc} with respect to each other are indicative of the cardiac abnormality.

The value-range of Sᵣₒₜ/S_{acc} for healthy cases can be selected to be e.g. a limit value Q and above, i.e. aortic stenosis is deemed to be present if Sᵣₒₜ/S_{acc}<Q, where Q is based on empirical data gathered from a group of patients and healthy persons. The limit value Q is not necessary constant, but the limit value Q can be changing according to an individual under consideration, according to time, and/or according to some other factors. Depending on the accelerometer and on the gyroscope, the limit value Q can be for example 1, 2, 3, 4, 5, 6, 7, 8, 9, 10, 11, 12, 13, 14, 15, 16, 17, 18, 19, 20, 21, 22, 23, 24, 25, or more than 25. Correspondingly, the value-range of Sᵣₒₜ/S_{acc} for healthy cases can be for example 1 and above, 2 and above, 3 and above, 4 and above, 5 and above, 6 and above, 7 and above, 8 and above, 9 and above, 10 and above, 11 and above, 12 and above, 13 and above, 14 and above, 15 and above, 16 and above, 17 and above, 18 and above, 19 and above, 20 and above, 21 and above, 22 and above, 23 and above, 24 and above, 25 and above, or more than 25 and above.

Depending on the accelerometer, on the gyroscope, and on the way of forming the first and second descriptor values Sᵣₒₜ and S_{acc}, the limit Q can be a positive value, zero, or a negative value. For example, depending on case, the value range of (αSᵣₒₜ+βS_{acc})/S_{acc} can be zero or above, zero or below, a positive number or above, a positive number or below, a negative number or above, or a negative number or below, where a and B are constant factors.

In an apparatus according to an exemplifying and non-limiting embodiment, the processing system 102 is configured to maintain value-ranges each representing a specific probability of cardiac abnormality, e.g. aortic stenosis. The processing system 102 is configured to set, in response to a situation in which the ratio Sᵣₒₜ/S_{acc} of the first and second descriptor values belongs to one or more of the value-ranges, the indicator signal to express a highest one of the probabilities of cardiac abnormality related to these one or more of the value-ranges. The value-ranges can be defined for example as follows:
value-range₁: 0<Sᵣₒₜ/S_{acc}<R₁, the probability of aortic stenosis is P₁%,
value-range₂: 0<Sᵣₒₜ/S_{acc}<R₂>R₁, the probability of aortic stenosis is P₂%<P₁%,
value-range₃: 0<Sᵣₒₜ/S_{acc}<R₃>R₂, the probability of aortic stenosis is P₃%<P₂%, and
value-range₄: 0<Sᵣₒₜ/S_{acc}<R₄>R₃, the probability of aortic stenosis is P₄%<P₃%.

Each of R₁, R₂, R₃, and R₄, can be based on empirical data gathered from a group of patients and healthy persons. Correspondingly, Each of P₁, P₂, P₃, and P₄, can be based on empirical data gathered from the group of patients and healthy persons. One or more of the values R₁, R₂, R₃, and R₄ is/are not necessarily constant or constants, but the one or more of these values can be changing according to an individual under consideration, according to time, and/or according to some other factors. Depending on the accelerometer and on the gyroscope, R₁ can be e.g. 1, 2, 3, 4, 5, 6, 7, 8, 9, 10, 11, 12, 13, 14, 15, 16, 17, 18, 19, 20, 21, 22, 23, 24, 25 or more than 25. Correspondingly, R₂ can be e.g. R₁+1, R₁+2, . . . , or R₁+more than 25, R₃ can be e.g. R₂+1, R₂+2, . . . , or R₂+more than 25, R₃ can be e.g. R₂+1, R₂+2, . . . , or R₂+more than 25, and R₄ can be e.g. R₃+1, R₃+2, . . . , or R₃+more than 25.

In an apparatus according to an exemplifying and non-limiting embodiment, the processing system 102 is configured to set the indicator signal outputted by the apparatus to express presence of aortic stenosis in response to the situation in which the first and second descriptor values Sᵣₒₜ and S_{acc} with respect to each other are indicative of the cardiac abnormality, for example the ratio Sᵣₒₜ/S_{acc} of the first and second descriptor values is detected to be outside the predetermined value-range corresponding to the healthy cases.

The processing system 102 can be implemented for example with one or more processor circuits, each of which can be a programmable processor circuit provided with appropriate software, a dedicated hardware processor such as, for example, an application specific integrated circuit “ASIC”, or a configurable hardware processor such as, for example, a field programmable gate array “FPGA”. The memory 106 can be implemented for example with one or more memory circuits, each of which can be e.g. a random-access memory “RAM” device.

A first computer-implemented system according to an exemplifying and non-limiting embodiment for detecting a cardiac abnormality or a risk of having a cardiac abnormality in an individual comprises:
a sensor apparatus for contacting a chest of the individual and configured to measure motion of the chest of the individual, and
a processing system configured to i) calculate one or more parameters relating to the cardiac abnormality from the measured motion of the chest of the individual and ii) detect a presence or a risk of having the cardiac abnormality based on the calculated one or more parameters, wherein the cardiac abnormality comprises aortic stenosis.

In the first computer-implemented system according to an exemplifying and non-limiting embodiment, the sensor apparatus further comprises an accelerometer, a gyroscope, or both.

In the first computer-implemented system according to an exemplifying and non-limiting embodiment, the sensor apparatus is provided in a smartphone.

In the first computer-implemented system according to an exemplifying and non-limiting embodiment, the measuring the motion of the chest of the individual comprises measuring an acceleration, a rotation, or both of a heart of the individual from the sensor apparatus placed on a chest of the individual.

In the first computer-implemented system according to an exemplifying and non-limiting embodiment, the one or more parameters describe cardiac acceleration, cardiac rotation, or both.

In the first computer-implemented system according to an exemplifying and non-limiting embodiment, the cardiac abnormality further comprises heart valve disease, heart failure, atrial fibrillation, or any combination thereof.

A second computer-implemented system according to an exemplifying and non-limiting embodiment for detecting a cardiac abnormality or a risk of having a cardiac abnormality in an individual comprises:
a processing system configured to process accelerometer signal and gyroscope signal, wherein the processing comprises comparing the accelerometer signal and the gyroscope signal to one or more adjustable predetermined cardiac abnormality threshold data values, and
an output configured to make a signal available to a user, wherein the signal comprises an indication that the accelerometer signal and the gyroscope signal are above the one or more cardiac abnormality threshold data values.

The second computer-implemented system according to an exemplifying and non-limiting embodiment further comprises a sensor apparatus.

In the second computer-implemented system according to an exemplifying and non-limiting embodiment, the sensor apparatus further comprises an accelerometer, a gyroscope, or both.

In the second computer-implemented system according to an exemplifying and non-limiting embodiment, the accelerometer signal and the gyroscope signal are collected from the chest movement of the individual.

In the second computer-implemented system according to an exemplifying and non-limiting embodiment, the accelerometer signal further comprises one or more properties of a time-trend of acceleration measured from the individual.

In the second computer-implemented system according to an exemplifying and non-limiting embodiment, the gyroscope signal further comprises one or more properties of a time-trend of rotation measured from the individual.

In the second computer-implemented system according to an exemplifying and non-limiting embodiment, the processing system is further configured to compare the gyroscope signal to the accelerometer signal to produce a ratio output indicative of a ratio of the gyroscope signal to the accelerometer signal.

In the second computer-implemented system according to an exemplifying and non-limiting embodiment, the processing system is further configured to compare the one or more threshold values to the ratio output.

In the second computer-implemented system according to an exemplifying and non-limiting embodiment, the output further comprises an indication of a presence of cardiac abnormality, an indication of a risk of the cardiac abnormality, or an indication to contact a healthcare provider.

In the second computer-implemented system according to an exemplifying and non-limiting embodiment, the cardiac abnormality comprises aortic stenosis.

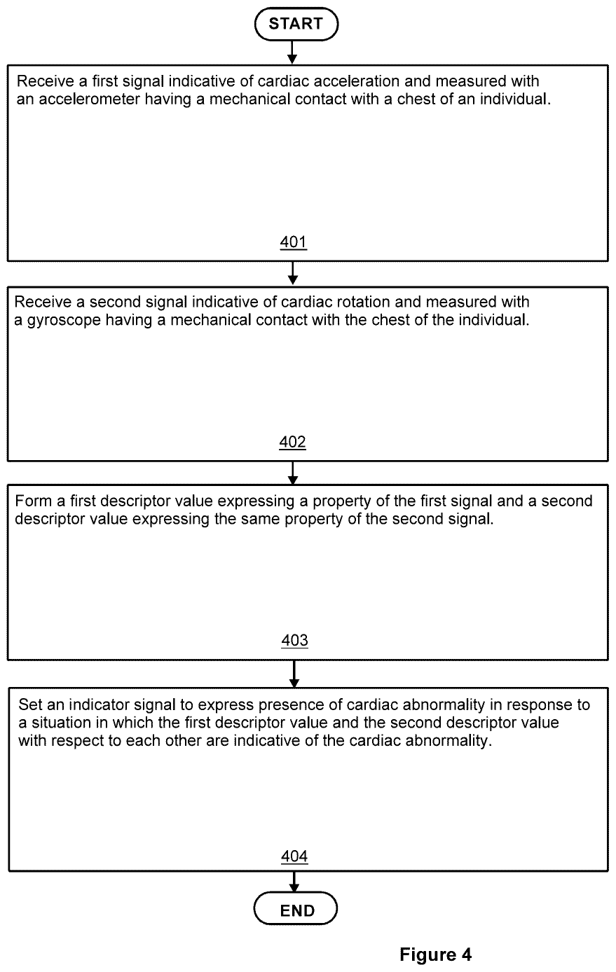

FIG. 4 shows a flow chart of a method according to an exemplifying and non-limiting embodiment for producing information indicative of cardiac abnormality, such as aortic stenosis, heart valve disease, heart failure, and/or atrial fibrillation. The method comprises the following actions:
action 401: receiving a first signal indicative of cardiac acceleration and measured with an accelerometer having a mechanical contact with a chest of an individual,
action 402: receiving a second signal indicative of cardiac rotation and measured with a gyroscope having a mechanical contact with the chest of the individual,
action 403: forming a first descriptor value expressing a property of the first signal and a second descriptor value expressing the same property of the second signal, and
action 404: setting an indicator signal to express presence of cardiac abnormality in response to a situation in which the first descriptor value and the second descriptor value with respect to each other are indicative of the cardiac abnormality.

The indicator signal can express an instruction to contact a healthcare provider. Alternatively or in combination, the indicator signal can be transmitted to a healthcare provider to indicate the presence of cardiac abnormality. The indicator signal can be cardiac measurement data transmitted to a healthcare provider.

A method according to an exemplifying and non-limiting embodiment comprises determining whether a ratio of the first and second descriptor values is outside a predetermined value-range corresponding to healthy cases in order to determine whether the first and second descriptor values with respect to each other are indicative of the cardiac abnormality.

A method according to an exemplifying and non-limiting embodiment comprises measuring the above-mentioned first and second signals with the accelerometer and the gyroscope from the chest of the individual. A method according to another exemplifying and non-limiting embodiment comprises reading these signals from a memory, in which case the signals have been measured earlier and recorded in the memory. A method according to an exemplifying and non-limiting embodiment comprises receiving the signals from an external data transfer system. Therefore, the measuring is not an essential and necessary step of methods according to embodiments of the invention.

In a method according to an exemplifying and non-limiting embodiment, the first descriptor value is proportional to strength of the first signal, and the second descriptor value is proportional to strength of the second signal. The first descriptor value can be proportional to e.g. power, energy, an amplitude, or the like of the first signal. Correspondingly, the second descriptor value can be proportional to e.g. power, energy, an amplitude, or the like of the second signal.

In a method according to an exemplifying and non-limiting embodiment, the first descriptor value is proportional to a median value of peak values of the first signal on successive heart-beat periods, and the second descriptor value is proportional to a median value of peak values of the second signal on the successive heart-beat periods.

In a method according to an exemplifying and non-limiting embodiment, the first descriptor value is proportional to an arithmetic average of peak values of the first signal on successive heart-beat periods, and the second descriptor value is proportional to an arithmetic average of peak values of the second signal on the successive heart-beat periods.

In a method according to an exemplifying and non-limiting embodiment, the first descriptor value is proportional to a peak-to-average ratio of the first signal and the second descriptor value is proportional to a peak-to-average ratio of the second signal.

A method according to an exemplifying and non-limiting embodiment comprises setting the indicator signal to express presence of aortic stenosis in response to the situation in which the ratio of the first and second descriptor values is outside the predetermined value-range corresponding to the healthy cases.

A method according to an exemplifying and non-limiting embodiment comprises maintaining value-ranges each representing a specific probability of cardiac abnormality, e.g. aortic stenosis. The method comprises setting, in response to a situation in which the ratio of the first and second descriptor values belongs to one or more of the value-ranges, the indicator signal to express a highest one of the probabilities of cardiac abnormality related to these one or more of the value-ranges.

A first computer-implemented method according to an exemplifying and non-limiting embodiment for detecting a cardiac abnormality or a risk of having a cardiac abnormality in an individual comprises:
measuring, with a sensor apparatus placed on a chest of the individual, motion of a chest of the individual,
calculating one or more parameters related to the cardiac abnormality from the measured motion of the chest of the individual, and
detecting a presence or a risk of having the cardiac abnormality based on the calculated one or more parameters, wherein the cardiac abnormality comprises aortic stenosis.

In the first computer-implemented method according to an exemplifying and non-limiting embodiment, the sensor apparatus comprises an accelerometer, a gyroscope, or both.

In the first computer-implemented method according to an exemplifying and non-limiting embodiment, the sensor apparatus is provided in a smartphone.

In the first computer-implemented method according to an exemplifying and non-limiting embodiment, the measuring the motion of the chest of the individual comprises measuring an acceleration, a rotation, or both of a heart of the individual from the sensor apparatus placed on the chest.

In the first computer-implemented method according to an exemplifying and non-limiting embodiment, the one or more parameters describe cardiac acceleration, cardiac rotation, or both.

In the first computer-implemented method according to an exemplifying and non-limiting embodiment, the cardiac abnormality further comprises heart valve disease, heart failure, atrial fibrillation, or any combination thereof.

A second computer-implemented method according to an exemplifying and non-limiting embodiment for detecting a cardiac abnormality or a risk of having a cardiac abnormality in an individual comprises:
processing accelerometer signal and gyroscope signal, wherein the processing comprises comparing the accelerometer signal and the gyroscope signal to one or more adjustable predetermined cardiac abnormality threshold data values, and
outputting a signal to the user, wherein the signal comprises an indication that the accelerometer signal and the gyroscope signal are above the one or more cardiac abnormality threshold data values.

The second computer-implemented method according to an exemplifying and non-limiting embodiment further comprises collecting the accelerometer signal and the gyroscope signal using a sensor apparatus.

In the second computer-implemented method according to an exemplifying and non-limiting embodiment, the accelerometer signal and the gyroscope signal are collected from the chest movement of the individual.

In the second computer-implemented method according to an exemplifying and non-limiting embodiment, the accelerometer signal further comprises one or more properties of a time-trend of acceleration measured from the individual.

In the second computer-implemented method according to an exemplifying and non-limiting embodiment, the gyroscope signal further comprises one or more properties of a time-trend of rotation measured from the individual.

In the second computer-implemented method according to an exemplifying and non-limiting embodiment, the processing further comprises comparing the gyroscope signal to the accelerometer signal to produce a ratio output indicative of a ratio of the gyroscope signal to the accelerometer signal.

The second computer-implemented method according to an exemplifying and non-limiting embodiment further comprises comparing the one or more threshold values to the ratio output.

The second computer-implemented method according to an exemplifying and non-limiting embodiment further comprises outputting an indication of a presence of cardiac abnormality, an indication of a risk of cardiac abnormality, or an indication to contact a healthcare provider.

In the second computer-implemented method according to an exemplifying and non-limiting embodiment, the cardiac abnormality comprises aortic stenosis.

A computer program according to an exemplifying and non-limiting embodiment comprises computer executable instructions for controlling a programmable processing system to carry out a method according to any of the above-presented exemplifying and non-limiting embodiments.

A computer program according to an exemplifying and non-limiting embodiment comprises software modules for producing information indicative of cardiac abnormality, such as aortic stenosis, heart valve disease, heart failure, and/or atrial fibrillation. The software modules comprise computer executable instructions for controlling a programmable processing system to:
receive a first signal indicative of cardiac acceleration and measured with an accelerometer having a mechanical contact with a chest of an individual,
receive a second signal indicative of cardiac rotation and measured with a gyroscope having a mechanical contact with the chest of the individual,
form a first descriptor value expressing a property of the first signal and a second descriptor value expressing the same property of the second signal, and
set an indicator signal to express presence of cardiac abnormality in response to a situation in which the first descriptor value and the second descriptor value with respect to each other are indicative of the cardiac abnormality, e.g. a ratio of the first descriptor value and the second descriptor value is outside a predetermined value-range corresponding to healthy cases.

The software modules can be e.g. subroutines or functions implemented with a suitable programming language and with a compiler suitable for the programming language and for the programmable processing system under consideration. It is worth noting that also a source code corresponding to a suitable programming language represents the computer executable software modules because the source code contains the information needed for controlling the programmable processing system to carry out the above-presented actions and compiling changes only the format of the information. Furthermore, it is also possible that the programmable processing system is provided with an interpreter so that a source code implemented with a suitable programming language does not need to be compiled prior to running.

A computer program product according to an exemplifying and non-limiting embodiment comprises a computer readable medium, e.g. a compact disc (“CD”), encoded with a computer program according to an embodiment of invention.

A computer readable medium according to an exemplifying and non-limiting embodiment is encoded with a computer program according to an embodiment of invention.

A computer readable medium, such as computer-executable code, may take many forms, including but not limited to, a tangible storage medium, a carrier wave medium or physical transmission medium. Non-volatile storage media include, for example, optical or magnetic disks, such as any of the storage devices in any one or more computers or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency “RF” and infrared “IR” data communications. Common forms of computer-readable media therefore include for example: a floppy disk, a flexible disk, hard disk, magnetic tape, any other magnetic medium, a compact disc read only memory “CD-ROM”, digital video disc “DVD” or DVD-ROM, any other optical medium, punch cards paper tape, any other physical storage medium with patterns of holes, a random access memory “RAM”, a read only memory “ROM”, a programmable read only memory “PROM” and an erasable programmable read only memory “EPROM”, a flash-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

A signal according to an exemplifying and non-limiting embodiment is encoded to carry information defining a computer program according to an embodiment of invention.

### EXAMPLES

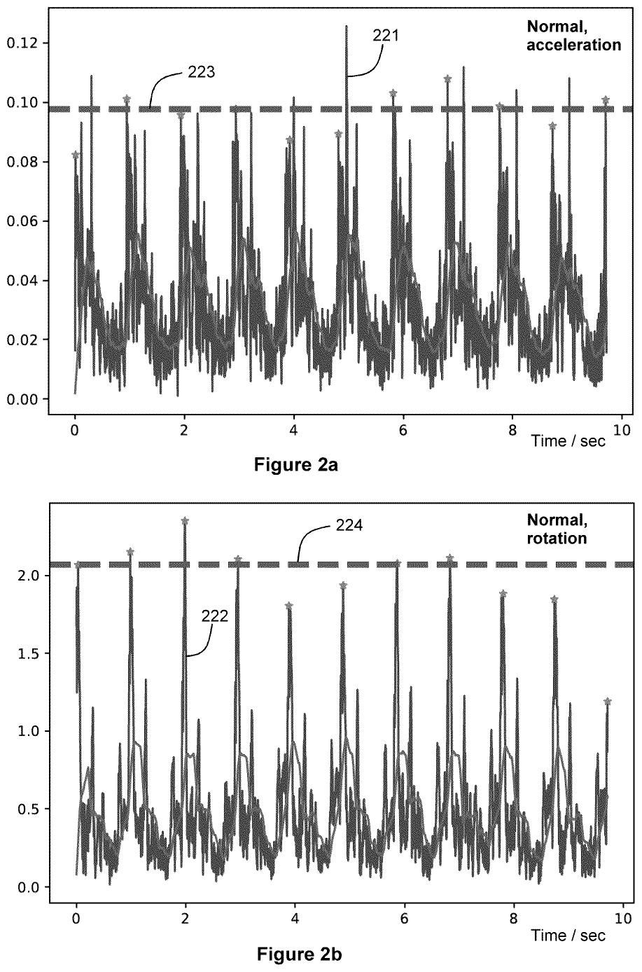

FIG. 2a illustrates a waveform of a first signal 221 indicative of cardiac acceleration and FIG. 2b illustrates a waveform of a second signal 222 indicative of cardiac rotation in a normal case when an individual under consideration is at rest. In the exemplifying case shown in FIG. 2a, the first signal 221 is measured with a three-axis accelerometer and the first signal 221 at a time moment ti is defined as:

$$\sqrt{( {a}_{x i}^{2}} + {a}_{yi}^{2} + {a}_{z i}^{2} ) , \qquad ( 11 )$$

where i is an index increasing with time, aₓᵢ is an i^{th} sample of an x-directional component of the first signal i.e. an x-directional component of the cardiac acceleration in a cartesian coordinate system, a_{yi} is an i^{th} sample of a y-directional component of the first signal in the cartesian coordinate system, and a_{zi} is an i^{th} sample of a z-directional component of the first signal in the cartesian coordinate system. The z-direction is towards the chest of the individual as illustrated with the coordinate system 199 in FIG. 1. The second signal 222 is measured with a three-axis gyroscope, and the second signal 222 at the time moment tᵢ is defined as:

$$\sqrt{( {ω}_{x i}^{2}} + {ω}_{y i}^{2} + {ω}_{z i}^{2} ) , \qquad ( 12 )$$

where ωₓᵢ is an i^{th} sample of an x-directional component of the second signal i.e. the cardiac rotation with respect to the x-direction of the cartesian coordinate system, ω_{yi} is an i^{th} sample of an y-directional component of the second signal in the cartesian coordinate system, and ω_{zi} is an i^{th} sample of an z-directional component of the second signal in the cartesian coordinate system.

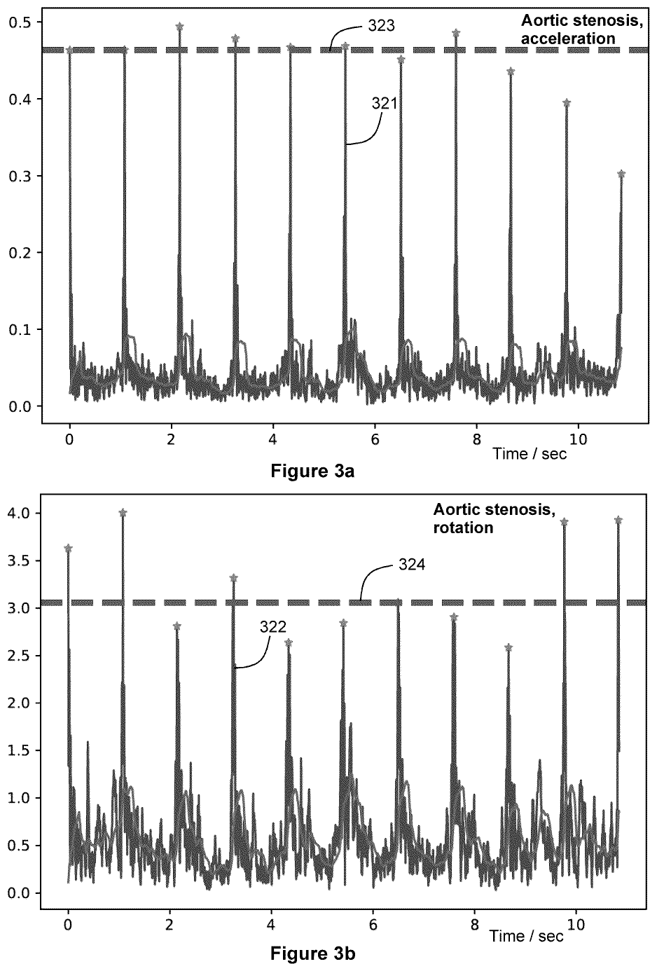

FIG. 3a illustrates a waveform of a first signal 321 indicative of cardiac acceleration and FIG. 3b illustrates a waveform of a second signal 322 indicative of cardiac rotation in a case of aortic stenosis when an individual under consideration is at rest. The first and second signals 321 and 322 are measured with a three-axis accelerometer and a three-axis gyroscope and are defined according to the above-presented formulas 11 and 12, respectively.

In FIGS. 2a-3b, dashed lines 223, 224, 323, and 324 denote the median values of peak values of the first and second signals on successive heart-beat periods, respectively. As discussed above, these median values can be used as the first and second descriptor values Sᵣₒₜ and S_{acc}. The median values are advantageous since they are tolerant against noise and outliers.

The ratio of the median values shown in FIGS. 2a and 3a and related to the cardiac acceleration is 0.46/0.099≈4.6, whereas the ratio of the median values shown in FIGS. 2b and 3b and related to the cardiac rotation is 3.06/2.07≈1.48. Thus, as shown in FIGS. 2a, 2b, 3a, and 3b, the aortic stenosis increases the median value of the peaks of the cardiac acceleration more strongly than the median value of the peaks of the cardiac rotation. Thus, the above-mentioned ratio Sᵣₒₜ/S_{acc} can be used as an indicator of aortic stenosis.

In the exemplifying normal case illustrated in FIGS. 2a and 2b, the ratio Sᵣₒₜ/S_{acc} is 2.07/0.099≈20.9. In the exemplifying aortic stenosis case illustrated in FIGS. 3a and 3b, the corresponding ratio Sᵣₒₜ/S_{acc} is 3.06/0.46≈6.65. The value-range of Sᵣₒₜ/S_{acc} for healthy cases can be selected to be e.g. 14 and above, i.e. aortic stenosis is deemed to be present if Sᵣₒₜ/S_{acc}<14.

FIGS. 2a-3b also show that the aortic stenosis increases the peak-to-average ratio of the cardiac acceleration, FIGS. 2a and 3a, more strongly than the peak-to-average ratio of the cardiac rotation, FIGS. 2b and 3b. Thus, the first and second descriptor values Sᵣₒₜ and S_{acc} can be the peak-to-average ratios of the first and second signals.

As mentioned earlier in this document, one can define value-ranges each representing a specific probability of cardiac abnormality, e.g. aortic stenosis, and, in response to a situation in which the ratio of the first and second descriptor values belongs to one or more of the value-ranges, the indicator signal can be set to express a highest one of the probabilities of cardiac abnormality related to these one or more of the value-ranges. In the exemplifying case illustrated in FIGS. 2a-3b, the above-mentioned value-ranges can be defined for example as follows:
value-range₁: 0<Sᵣₒₜ/S_{acc}<7, the probability of aortic stenosis is P₁%,
value-range₂: 0<Sᵣₒₜ/S_{acc}<10, the probability of aortic stenosis is P₂%<P₁%,
value-range₃: 0<Sᵣₒₜ/S_{acc}<14, the probability of aortic stenosis is P₃%<P₂%, and
value-range₄: 0<Sᵣₒₜ/S_{acc}<18, the probability of aortic stenosis is P₄%<P₃%.

### REMARKS

The specific examples provided in the description given above should not be construed as limiting the scope and/or the applicability of the invention. Lists and groups of examples provided in the description given above are not exhaustive unless otherwise explicitly stated.

What is claimed is:

1. A computer-implemented method for determining a cardiac abnormality of a subject, The computer-implemented method comprising:
(a) obtaining a first signal indicative of cardiac acceleration;
(b) obtaining a second signal indicative of cardiac rotation;
(c) determining a first value relating to a property of the first signal and a second value relating to a property of the second signal; and
(d) generating an indication of cardiac abnormality of the subject based at least in part on a comparison between the first value and the second value.

2. The computer-implemented method of claim 1, wherein obtaining the first signal indicative of the cardiac acceleration comprises measuring the first signal using an accelerometer in mechanical contact with a chest of the subject.

3. The computer-implemented method of claim 2, wherein obtaining the first signal indicative of the cardiac acceleration comprises measuring the first signal using a sensor system comprising the accelerometer.

4. The computer-implemented method of claim 3, wherein the sensor system is part of a smartphone.

5. The computer-implemented method of claim 1, wherein obtaining the second signal indicative of the cardiac acceleration comprises measuring the second signal using a gyroscope in mechanical contact with the chest of the individual.

6. The computer-implemented method of claim 5, wherein obtaining the second signal indicative of the cardiac acceleration comprises measuring the second signal using a sensor system comprising the gyroscope.

7. The computer-implemented method of claim 6, wherein the sensor system is part of a smartphone.

8. The computer-implemented method of claim 1, wherein the first value is proportional to a signal strength of the first signal, and the second value is proportional to a signal strength of the second signal.

9. The computer-implemented method of claim 8, wherein the first value is proportional to a signal power of the first signal, and the second value is proportional to a signal power of the second signal.

10. The computer-implemented method of claim 8, wherein the first value is proportional to a signal energy of the first signal, and the second value is proportional to a signal energy of the second signal.

11. The computer-implemented method of claim 8, wherein the first value is proportional to a median value of two or more peak values of the first signal, wherein the two or more peak values relate to peaks of successive heart-beat periods, and the second value is proportional to a median value of two or more peak values of the second signal, wherein the two or more peak values relate to the peaks of the successive heart-beat periods.

12. The computer-implemented method of claim 8, wherein the first value is proportional to an average of two or more peak values of the first signal, wherein the two or more peak values relate to peaks of successive heart-beat periods, and the second value is proportional to an average of two or more peak values of the second signal, wherein the two or more peak values relate to the successive heart-beat periods.

13. The computer-implemented method of claim 1, wherein the comparison between the first value and the second value comprises a ratio of the first value and the second value.

14. The computer-implemented method of claim 13, further comprising comparing the ratio to one or more value ranges, thereby generating a second comparison.

15. The computer-implemented method of claim 14, wherein generating the indication of the cardiac abnormality of the subject is based at least in part on the second comparison.

16. The computer-implemented method of claim 13, further comprising determining a plurality of value ranges, wherein a value range of the plurality of value ranges relates to a risk percentage of the cardiac abnormality.

17. The computer-implemented method of claim 16, further comprising comparing the ratio to at least a portion of the plurality of value ranges.

18. The computer-implemented method of claim 17, wherein generating the indication of the cardiac abnormality of the subject is based at least in part on the comparing.

19. The computer-implemented method of claim 1, wherein the cardiac abnormality is the aortic stenosis.

20. The computer-implemented method of claim 1, wherein the property of the first signal and the property of the second signal are the same.

21. A system for determining a cardiac abnormality of a subject, the system comprising:
(a) a signal interface configured to obtain a first signal indicative of a cardiac acceleration and a second signal indicative of a cardiac rotation; and
(b) a processor configured to receive information from the signal interface, wherein the processor is further configured to:
(i) determine a first value relating to a property of the first signal;
(ii) determine a second value relating to a property of the second signal; and
(iii) generate an indication of the cardiac abnormality of the subject based at least in part on a comparison between the first value and the second value.
